(19) United States
(12) Patent Application Publication
(10) Pub. No.: US 2005/0137167 A1
(43) Pub. Date: Jun. 23, 2005

(54) Derivatives of partially desulphated glycosaminoglycans as heparanase inhibitors, endowed with antiangiogenic activity and devoid of anticoagulating effect

(76) Inventors: Benito Casu, Milan (IT); Giangiacomo Torri, Milan (IT); Annamaria Naggi, Milan (IT); Claudio Pisano, Rome (IT); Giuseppe Giannini, Rome (IT); Sergio Penco, Rome (IT)

Correspondence Address: NIXON & VANDERHYE, PC, 1100 N GLEBE ROAD, 8TH FLOOR, ARLINGTON, VA, 22201-4714, US

(21) Appl. No.: 10/489,359

(22) PCT Filed: Sep. 12, 2001
(86) PCT No.: PCT/IT01/00472

(51) Int. Cl. C08B 37/10; C07H 5/04; A61K 31/727

(52) U.S. Cl. 514/56; 536/21; 536/55.3

(57) ABSTRACT

Partially desulphated glycosaminoglycan derivatives are described, particularly heparin, and more particularly formula (I) compounds where the U, R and R₁ groups have the meanings indicated in the description. Said glycosaminoglycan derivatives are endowed with antiangiogenic and heparanase-inhibiting activity ans are devoid of anticoagulant activity.

The invention described herein relates to partly desulphated glycosaminoglycan derivatives, particularly heparins, to processes for their preparation, to their use as active ingredients for the preparation of medicaments useful in pathological conditions, like tumors, included the metastatic forms, and for any therapeutic indication gaining benefit from the inhibition of the heparanase, and to pharmaceutical compositions containing them.

### State of the Art

Studies performed in the Tumor Biological Research Unit of the Hadassah-Hebrew University Hospital-Israel (Isr. Med. Assoc. J. 2000, 2, 37-45; J. Med. Chem. 2000, 43, 2591-600; Invasion Metastasis 1994-95, 14, 290-302; Exp. Cell Res. 1992, 201, 208-15;) focus on the involvement of heparin-binding growth factors, heparan sulphate and heparan sulphate-degrading enzymes (heparanase) in tumor angiogenesis and metastasis. These studies have been applied to screening and to the identification of heparin derivatives and heparin/heparan sulphate mimetics with potent heparanase inhibiting activity (Nature Med. 1999, 5, 735-6; Science, 1999, 285, 33-4].

Tumor cells release the enzyme heparanase, an endo-β-D-glucuronidase which degrades the polysaccharide chain of heparan sulphate proteoglycans on cell surfaces and in the extracellular matrix.

Involvement in tumor angiogenesis of heparanase has been correlated with the ability to release bFGF (FGF-2) and other growth factors from its storage within the ECM (extracellular matrix). These growth factors provide a mechanism for induction of neovascularization in normal and pathological situations.

Heparanase may thus facilitate not only tumor cell invasion and metastasis but also tumor angiogenesis, both critical steps in tumor progression.

Specific inhibitors of the heparanase enzyme prevent release and activation of growth factors stored by heparan sulphate as well as disruption of the ECM, and are regarded as a very promising approach to develop anticancer drugs.

So, one of possible therapeutic approaches for an antiangiogenic drug is the development of a potent and selective heparanase inhibitor.

For a discussion of angiogenesis, reference may be made to WO 01/55221, in the name of the present applicant.

Another important involvement of heparanase is both inflammation and autoimmunity. In fact, heparanase activity correlates also with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of heparan sulphate by heparanase activity. The enzyme is released from intracellular compartments (i.e. lysosomes, specific granules) in response to various activation signals, suggesting its regulated involvement and presence in inflammatory sites and autoimmune lesions. Treatment of experimental animals with heparanase inhibitors (i.e., non-anticoagulant species of low molecular weight heparin—LMWH) markedly reduced the incidence of experimental autoimmune encephalomyelitis (EAE), adjuvant arthritis and graft rejection in experimental animals, indicating that heparanase inhibitors may be applied to inhibit autoimmune and inflammatory disease.

Heparin

Heparin is a heterogeneous mixture of naturally occurring polysaccharides of various lengths and various degrees of sulphation which possesses anticoagulant activity and is secreted by the connective tissue mast cells present in the liver (from which it was first isolated), in the muscles, lungs, thymus and spleen.

In addition to the regular sequence, a sequence corresponding to the active site for antithrombin activity has been identified in heparin.

The antitumor and antimetastatic activity of heparin and its derivatives is due to its ability to inhibit heparanase, to block growth factors and to regulate angiogenesis.

Heparan Sulphates (HS)

Heparan sulphates (HS) are ubiquitous protein ligands. The proteins bind to the HS chains for a variety of actions from simple immobilisation or protection against the proteolytic degradation action to specific modulations of biological activities, such as angiogenesis.

The carbohydrate skeleton, in both heparin and the heparan sulphates (HS), consists in an alternation of D-glucosamine (GlcN) and hexuronic acids (GIcA or IdoA).

In heparin, the GlcN residues are mainly N-sulphated, whereas in HS they are both N-sulphated and N-acetylated, with a small amount of unsubstituted NH₂ groups.

HS is also on average less O-sulphated than heparin.

The use of heparin in the treatment of angiogenesis disorders, such as tumours, particularly metastases, is substantially limited by the anticoagulant activity of heparin.

Chemical modifications have been made to heparin so as to reduce its anticoagulant capacity, at the same time preserving its antitumor properties.

The opening of a unit of glucuronic acid in the antithrombin site reduces the affinity of heparin for antithrombin: in this way, heparins can be used with reduced anticoagulant effects, but still retaining antiangiogenic properties.

Heparanases

Heparanases are enzymes belonging to a family of endoglycosidases (an endo-β-D-glucuronidase) that hydrolyse the internal glycoside bonds of the chains of heparan sulphates (HS) and heparin.

These endoglycosidases are involved in the proliferation of tumour cells, in metastases and in the neovascularisation of tumours. These enzymes are biological targets for antiangiogenic activity. In the scientific literature there are a large number of structure/activity correlation studies (see, for example, Lapierre F. et al., Glycobiology, vol. 6, (3), 355-366, 1996). Though many aspects have still to be clarified, studies have been reported regarding the inhibition of heparanases by heparin and its derivatives, using specific tests which have led to the emergence of considerations of a structural type which may serve as guides for obtaining new, more selective derivatives.

In the above-mentioned work of Lapierre et al., heparin derivatives are described as obtained by 2-O desulphation or by “glycol split” (oxidation with periodate and subsequent reduction with sodium borohydride). These derivatives, defined here as “2-O-desulphated heparin” and “RO-heparin”, respectively, have partly maintained the antiangiogenic activity of heparin as assessed by means of the CAM test in the presence of corticosteroids (ibid. page 360).

N-acyl heparin derivatives, which are closer mimics of heparan sulphate than heparin, have been reported to inhibit heparanase only somewhat less than N-sulphate derivatives. (Irimira T., Biochemistry 1986, 25, 5322-5328; Ishai-Michaeli R., et al, Biochemistry 1992, 31, 2080-2088).

Heparins and FGF

FGFs regulate multiple physiological processes such as cell growth and differentiation, but also functions involved in pathological processes such as tumour angiogenesis.

FGFs are growth factors (a family of more than 10 polypeptides, of which the acid (FGF-1) and basic FGFs (FGF-2) are the ones which have been most studied, which require a polysaccharide cofactor, heparin or HS, to bind to the FGF receptor (FGFR) and activate it.

Though the precise mechanism whereby heparin and HS activate FGFs is unknown, it is known, however, that heparin/FGF/FGFR form a “trimolecular” or “ternary” complex.

One mechanism postulated is that heparin and HS induce so-called sandwich dimerisation of FGF, and the latter, thus dimerised, forms a stable complex with FGFR.

Antimetastatic Activity of Heparin Derivatives

The ability of a primary tumour to generate metastatic cells is perhaps the main problem facing anticancer therapy.

Heparin derivatives with a substantial ability to block heparanase seem to be equally capable of inhibiting angiogenesis both in primary tumours and in metastases.

In addition, the inhibition of heparanase reduces the migration ability of tumour cells from the primary tumour to other organs.

The antimetastatic activity in animal models has been found to correlate with the heparanase-inhibiting ability of heparin and heparin derivatives (Bitan M. et al, Isr. J. Med. Sci. 1995, 31, 106-108) as well as other sulphated polysaccharides (Miao, H. Q. et al, Int. J. Cancer 1999, 83, 424-431, and references therein). Studies on the molecular-weight dependence of the antimetastatic activity indicated that also very low-MW heparins (Sciumbata, T., et al, Invasion Metastasis 1996, 16, 132-143) and oligosaccharide polysulphates (Parish, C. R., et al, Cancer Res. 1999, 59, 3433-3441) retain significant antimetastatic activity. Although in general removal of N-sulphate groups (N-desulphation) decreases the antimetastatic potential of heparins, this activity is partially restored upon N-acylation (N-acetylation, N-hexanoylation (Bitan M., 1995), and N-succinylation (Sciumbata, T., 1996) of resulting free NH₂ groups. The antimetastatic activity of heparins was found to be inversely correlated to their degrees of O-sulphation. (Bitan M., 1995). However, selective 2-O-desulphation of iduronic acid residues did not involve a strong reduction of the antimetastatic activity of heparin (Lapierre, F., Glycobiology 1996, 6, 355-366).

In general, both the heparanase-inhibiting and the antimetastatic activity of heparins and other sulphated polysaccharides decrease with decreasing molecular weight and degree of sulphation (Bitan M., 1995; Parish, C. R., 1999). However, these activities also depend on the carbohydrate backbone of the polysaccharide (type of residues and position of glycosydic linkages) (Parish, C. R., 1999). Since the tridimensional structure of the active site of heparanase is not yet known, it is difficult to predict which polysaccharide backbones and sulphation patterns most effectively inhibit the enzyme.

On the basis of the present knowledge, the structural requirements of heparin-like molecules that favour the angiogenesis-inhibiting action can be grouped in two categories on the basis of the target one intends to block:
a) inhibition of heparanase: although this enzyme recognizes and cleaves heparin and HS sequences of at least eight monosaccharide units containing N-acyl-glucosamine-glucuronic acid (or N-sulphated glucosamine residues see, for example, D. Sandback-Pikas et al. J. Biol. Chem., 273, 18777-18780 (1998) and references cited), its inhibition can be efficiently accomplished by heparin fragments longer than tetradecasaccharide (Bitan M., 1995) or by extensively sulphated, shorter oligosaccharides, such as maltohexaose sulphate (MHS) and phosphomannopentaose sulphate (PI-88) (Parish, C. R., 1999). However, both long heparin fragments and heavily sulphated oligosaccharides are anticoagulant, a property that should be avoided for potential antimetastatic drugs;
b) inhibition of angiogenic growth factors (fibroblast type: FGF-1 and FGF-2; vascular endothelium type: VEGF; vascular permeability type: VPF): to this end the heparin-like compounds preferably have sequences at least five monosaccharide units long, containing 2-sulphated iduronic acid and glucosamine N,6-sulphated (see, for example, M. Maccarana et al. J. Biol. Chem., 268, 23989-23905 (1993)).

The literature discloses small peptides (5-13 amino acids) with antiangiogenic activity (U.S. Pat. No. 5,399,667 of the University of Washington) which act by binding to a thrombospondin receptor, or longer peptides (50 amino acids approx.).

Modified platelet factors are known—(EP 0 589 719, Lilly), capable of inhibiting endothelial proliferation, with IC₅₀=7 nM.

Oligosaccharide fragments with antiangiogenic activity have also been amply described: it has been found, in fact, that by varying the carbohydrate sequence the interaction selectivity can be increased.

In addition, heparin can be used as a vehicle for substances which are themselves antiangiogenic, such as some steroids, exploiting the affinity of heparin for vascular endothelial cells; see, for example, WO 93/18793 of the University of Texas and Imperial Cancer Research Technology, where heparins are claimed with acid-labile linkers, such as adipic acid hydrazine, bound to cortisol. The antiangiogenic effect of the conjugated molecules is greater than that of the unconjugated molecules, even when administered simultaneously.

In Biochim. Biophys. Acta (1996), 1310, 86-96, heparins bound to steroids (e.g. cortisol) are described with a hydrazone group in C-20 that present greater antiangiogenic activity than the two unconconjugated molecules.

EP 0 246 654 by Daiichi Sc. describes sulphated polysaccharides with antiangiogenic activity with Phase II studies. EP 0 394 971 by Pharmacia & Upjohn—Harvard Coll. describes hexa-saccharides—heparin fragments—with low sulphation, capable of inhibiting the growth of endothelial cells and angiogenesis stimulated by FGF-1. EP 0 618 234 by Alfa Wasserman describes a method for preparing semisynthetic glycosaminoglycans with a heparin or heparan structure bearing a nucleophilic group. WO 95/05182 by Glycomed describes various sulphated oligosaccharides with anticoagulant, antiangiogenic and anti-inflammatory activity. U.S. Pat. No. 5,808,021 by Glycomed describes a method for preparing substantially non-depolymerised 2-O, 3-O desulphated heparin with a desulphation percentage in positions 2—of the iduronic acid (I, 2-O) and in position 3 of the glucosamine unit (A, 3-O) ranging from approximately 99 to approximately 75% of the original percentage. This method envisages desulphation conducted in the presence of a cation of a bivalent metal, exemplified by calcium or copper, followed by lyophilisation of the product obtained. The desulphated heparins have antiangiogenic activity. EP 0 251 134, Yeda Res & Dev Co Ltd et al, discloses the use of subcoagulant dosages of heparin or its derivatives for preventing allograft rejection and treating autoitnmune diseases. The activity of heparin is given by inhibition of heparanase. WO 88/05301, Univ. Australian Nat., discloses antimetastatic and/or antiinflammatory compositions containing a sulphated polysaccharide, which is heparanase inhibitor. Heparin, fucoidan, pentosan sulphate, dextran sulphate are provided. WO 92/01003, Univ. Texas System, discloses the use of a heparin derivative, which is devoid of anticoagulation activity, as heparanase inhibitor. These derivatives have sulphamino or O-sulphate groups, M. W. 1000-15000 and each terminal monomeric unit is a monomeric repeating unit with a terminal O atom bound to a blocking group. WO 94/14851 and WO 96/06867, Glycomed, provide 2-O, 3-O-de-sulphated mucosal heparin, or fragments thereof, being at least 96.7% de-sulphated at the 2-O position and ate least 75% desulphated at the 3-O position useful as non-anticoagulant heparanase inhibitors. WO 95/09637 and WO 96/09828, Glycomed, discloses highly sulphated maltooligosaccharide compounds with heparin like properties. WO 95/30424, Glycomed, provides 6-O-desulphated heparin or fragments thereof with heparanase inhibiting activity. WO 96/33726, Univ. Australian Nat., discloses sulphated oligosaccharides as heparan mimetics having heparanase inhibiting activity. WO 01/35967, Knoll A G, provides a method for treating cardiac insufficiency and related conditions by administering an heparanase inhibitor, among which, heparin which has partly reduced COOH groups, or is at least partly N-desulphated and N-acetylated or is at least partly N,O-desulphated and N-resulphated or is O-acetylated is mentioned.

The aim of the invention described herein is to find optimal glycosaminoglycan structures for generating antiangiogenic activity based on heparanase inhibition and/or FGF growth factor inhibition mechanisms. An additional aim of the invention described herein is to provide a medicament with antiangiogenic activity which is essentially devoid of the typical side effects of heparin derivatives, such as, for example, anticoagulant activity.

WO 01/55221, in the name of the applicant, discloses glycosaminoglycans, particularly a desulphated heparin, with a desulphation degree not greater than 60% of the total uronic units. These derivatives are provided with antiangiogenic activity and are devoid of anticoagulant activity. Said compounds exert their antiangiogenic activity based on the inhibition of FGF. No activity was foreseen for inhibition of heparanase.

In quite general terms, WO 01/55221 also provides a modified heparin, containing glycosamine residues with different degrees of N-desulphation and optional subsequent total or partial acetylation. The general teaching of said reference does not explicitly describe the N-desulphation and optional subsequent total or partial acetylation steps.

### Abstract of the Invention

It has now been found that on subjecting a glycosaminoglycan, such as a heparin-like glycosaminoglycan, heparin or modified heparin, containing glucosamine residues with different degrees of N-desulphation and optional subsequent total or partial N-acylation (preferably N-acetylation), to controlled 2-O-desulphation treatment of the iduronic units up to a degree of desulphation not greater than 60% of the total uronic units, the growth-factor-mediated angiogenic properties are maintained.

Surprisingly, heparin 2-O-desulphated disclosed in the above mentioned WO 01/55221 are also inhibitors of heparanes. This property was found to be further enhanced upon glycol splitting of non-sulphated uronic acid residues. Glycol-splitting, a chemical modification leading to a dramatic loss of anticoagulant activity (Casu B., et al, Arzneim. Forsch. (Drug Res.) 1986, 36, 637-642) was also found to dramatically enhance the heparanase-inhibiting properties of partially N-acetylated heparins obtained through 50% N-desulphation followed by N-acetylation of the resulting free amino groups and of the 2-O-desulphated compounds.

The desulphation carried out in the conditions described in the present invention also produces the formation of iduronic units with an oxyranic ring in position 2,3. The opening of the oxyranic ring in the conditions described in the present invention gives rise to L-iduronic or L-galacturonic units.

It is an object of the invention described herein the use of said glycosaminoglycan derivatives for the preparation of a medicament having heparanase and/or FGF growth factor inhibiting activity.

According to the present invention, said glycosaminoglycan derivative is preferably a heparin-like glycosaminoglycan. Still according to the present invention, said glycosaminoglycan derivative is a modified heparin, containing glycosamine residues with different degrees of N-desulphation and optional subsequent total or partial N-acetylation.

In one particular embodiment, the invention described herein refers to a formula (I) compound 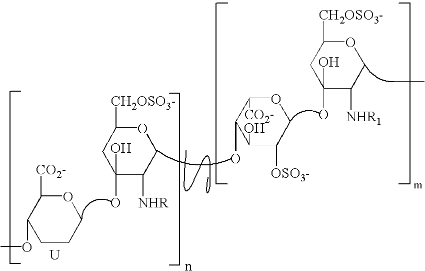
where the U ring may have the following meanings: 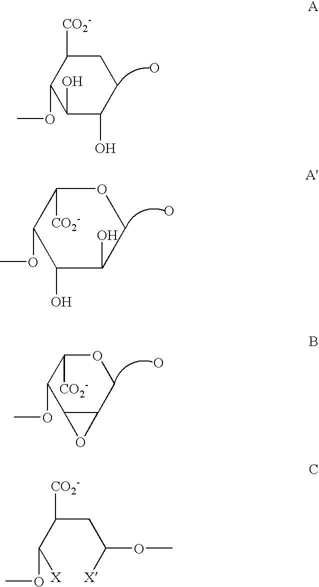
X and X′, which can be the same or different, are an aldehyde group or the —CH₂—D group, where D is hydroxyl or an amino acid, a peptide or a residue of a carbohydrate or oligosaccharide;
R and R₁, which can be the same or different, are an SO₃, a C₁-C₈ acyl residue, optionally bearing at least a further carboxy group; acetyl, hexanoyl, succinyl, pivaloyl are the preferred acyl residues;
n and m, which can be the same or different, may vary from 1 to 40; the sum of n+m ranges from 6 to 40; the m:n ratio ranges from 10:2 to 1:1;

The symbol [symbol] indicates that the units marked m and n are statistically distributed along the polysaccharide chain and are not necessarily in sequence.

Examples of C₁-C₈ acyl residue, optionally bearing at least a further carboxy group are acetyl, propionyl, butyryl, pentanoyl, hexanoyl, heptanoyl, and all the possible isomers, oxalyl, malonyl, succinyl, pivaloyl, glutaroyl; acetyl, hexanoyl, pivaloyl are the preferred acyl residues.

When R or R₁ are N-acyl groups, they preferentially range from 40 to 60% of the sum R+R₁. Preferably, m is greater than or equal to n. Preferably n ranges from 40 to 60% of the sum m+n.

The compounds of formula (I) above, wherein R and R₁ are C₁ or C₃-C₈ acyl residue are new.

The compounds which are the subject matter of the invention described herein, are characterized by a high power of inhibiting heparanase with interesting antiangiogenic properties, and are therefore useful as active ingredients for the preparation of medicaments for the treatment of pathologies gaining benefit from the inhibition of the heparanase, pathologies based on abnormal angiogenesis, and particularly for the treatment of metastases.

The compounds according to the present invention also inhibit FGFs.

Advantageously, the compounds according to the present invention show reduced, if not non-existent anticoagulant properties, thus avoiding or reducing the side effects typical of the heparins. A further advantage stems from the fact that the compounds according to the invention can be characterised with instrumental analytical techniques, such as NMR spectroscopy, thus allowing process control which is absolutely desirable from the industrial point of view.

Also in the case of modified heparins, molecular weight (MW) has a very important function when making angiogenesis inhibitors. It is well known, in fact, that a reduction in molecular weight (MW) up to values corresponding to penta-saccharide units does not lead to a loss of antiangiogenic activity. On the other hand, it has been established that, whereas beyond a certain length the heparin chains favour rather than inhibit activation of FGF, they are even better inhibitors of heparanase than shorter chains. However, the optimal chain length for inhibition of heparanase depends on the structure of the inhibitor (carbohydrate backbone, positional linkages, sulphation pattern) and should be established for any new type of potential inhibitors.

### DETAILED DESCRIPTION OF THE INVENTION

The compounds according to the present invention containing glycosamine residues with different degrees of N-desulphation and optional subsequent total or partial acetylation are herein specifically disclosed and claimed as new compounds.

What is meant by desulphation degree is the percentage of non-sulphated iduronic acids in relation to total uronic acids originally present in the starting heparin. One initial preferred range for the desulphation percentage is from approximately 40 to approximately 60%.

Among the formula (I) compounds, a first preferred compound is a heparin partially 2-O-desulphated with a molecular weight (MW) of 11200, a polydispersion index D of 1.3, a desulphation degree of 1.99 (expressed as the SO₃—:COO— molar ratio), a percentage of modified uronic acids compared to total uronic acids of approximately 50%. Said compound (hereinafter also called ST1514) is comprised in formula (I) where, among the other corresponding definitions, m:n=1:1 and the units marked m and n are distributed along the polysaccharide chain in a regular, alternating manner.

A second preferred compound is an LMW heparin partially 2-O-desulphated with a molecular weight (MW) of 3050, a polydispersion index of 2.2, a desulphation degree of 1.99 (expressed as the SO₃—:COO— molar ratio), a percentage of modified uronic acids compared to total uronic acids of approximately 50%. Said compound (hereinafter also called ST2010) is comprised in formula (I) where, among the other corresponding definitions, m:n=1:1 and the units marked m and n are distributed along the polysaccharide chain in a regular, alternating manner. This compound is obtained by nitrous acid depolymerization of ST1514, followed by reduction of aldehyde groups, therefore most of its reducing end residues are anhydromannose residues:

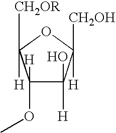

A third preferred compound is an LMW heparin partially 2-O-desulphated with a molecular weight of Mn=5800, Mw=7520, a polydispersion index of 1.294, a percentage of modified uronic acids compared to total uronic acids of approximately 50%. Said compound (hereinafter also called ST2184) is comprised in formula (I) where, among the other corresponding definitions, m:n=1:1 and the units marked m and n are distributed along the polysaccharide chain in a regular, alternating manner. This compound is obtained by nitrous acid depolymerization of ST1514, followed by reduction of aldehyde groups, therefore most of its reducing end residues are anhydromannose residues.

A fourth preferred compound is a partially N-desulphated and N-reacetylated heparin with a molecular weight (MW) of 11200, a polydispersion index of 1.3, a desulphation degree of 1.6 (expressed as the SO₃—:COO— molar ratio), a percentage of modified uronic acids compared to total uronic acids of approximately 30%. Said compound (hereinafter also called ST1518) is comprised in formula (I) where, among the other corresponding definitions, the 50% of the sum of R and R₁ is N-acetyl.

A fifth preferred compound is an LMW partially N-desulphated and N-reacetylated heparin with a molecular weight of Mn=4780, Mw=10000, a polydispersion index of 2.092, a percentage of modified uronic acids compared to total uronic acids of approximately 30%. Said compound (hereinafter also called ST2168) is comprised in formula (I) where, among the other corresponding definitions, the 50% of the sum of R and R₁ is N-acetyl.

A sixth preferred compound is a partially N-desulphated and N-reacetylated heparin with a molecular weight of Mn=10890, Mw=22370, a polydispersion index of 2.054. Said compound (hereinafter also called ST2037) is comprised in formula (I) where, among the other corresponding definitions, the 27% of the sum of R and R₁ is N-acetyl.

A seventh preferred compound is a partially N-desulphated and N-reacetylated heparin with a molecular weight of Mn=10210, Mw=21270, a polydispersion index of 2.083. Said compound (hereinafter also called ST2038) is comprised in formula (I) where, among the other corresponding definitions, the 39% of the sum of R and R₁ is N-acetyl.

An eighth preferred compound is a partially N-desulphated and N-reacetylated heparin with a molecular weight of Mn=11070, Mw=22000, a polydispersion index of 1.987. Said compound (hereinafter also called ST2041) is comprised in formula (I) where, among the other corresponding definitions, the 64% of the sum of R and R₁ is N-acetyl.

A ninth preferred compound is a partially N-desulphated and N-reacetylated heparin, a percentage of modified uronic acids compared to total uronic acids of approximately 30%. Said compound is comprised in formula (I) where, among the other corresponding definitions, the 27% of the sum of R and R₁ is N-acetyl (ST2185).

A tenth preferred compound is a partially N-desulphated and N-reacetylated heparin, a percentage of modified uronic acids compared to total uronic acids of approximately 30%. Said compound (hereinafter also called ST2186) is comprised in formula (I) where, among the other corresponding definitions, the 39% of the sum of R and R₁ is N-acetyl.

A eleventh preferred compound is a partially N-desulphated and N-reacetylated heparin, a percentage of modified uronic acids compared to total uronic acids of approximately 30%. Said compound (hereinafter also called ST2187) is comprised in formula (I) where, among the other corresponding definitions, the 64% of the sum of R and R₁ is N-acetyl.

A twelfth preferred compound is a partially 2-O-desulphated heparin with a molecular weight (MW) of 12900 D, a polydispersion index D of 1.5, a desulphation degree of 1.9 (expressed as SO₃—:COO— molar ratio), percentage of modified uronic acids compared to total uronic acids: 5% epoxide groups, 29% oxidated and reduced uronic residues. Said compound (hereinafter also called ST1513) is comprised in formula (I) where, among the other corresponding definitions, m:n=1:1 and the units marked m and n are distributed along the polysaccharide chain in a regular, alternating manner.

A thirteenth preferred compound is a partially 2-O-desulphated heparin with a molecular weight (MW) of 9200 D, a polydispersion index D of 1.5, percentage of modified uronic acids compared to total uronic acids: 11% epoxide groups, 27.5% oxidated and reduced uronic residues. Said compound (hereinafter also called ST1515) is comprised in formula (I) where, among the other corresponding definitions, m:n=1:1 and the units marked m and n are distributed along the polysaccharide chain in a regular, alternating manner.

A fourteenth preferred compound is a partially 2-O-desulphated heparin with a molecular weight (MW) of 11000 D, a polydispersion index D of 1.5, a desulphation degree of 1.93 (expressed as SO₃—:COO— molar ratio), a percentage of modified uronic acids compared to total uronic acids: 5% epoxide groups, 29% oxidated and reduced uronic residues.

The preparation of compound ST1514, ST1513, ST1516 and ST1515 are specifically disclosed in WO 01/55221.

The partially 2-O-desulphated derivatives according to the invention described herein are prepared as disclosed in the above mentioned WO 01/55221.

As far as the N-desulphated and optionally N-acetylated glycosaminoglycans according to the present invention, they can be prepared by means of a process, enabling also the preparation of the 2-O-partially desulphated heparins, comprising:

a) N-desulphation by solvolytic hydrolysis of sulphamino residues in DMSO:H₂O 95:5 v:v at ambient temperature for a time ranging from 0.5 to 8 h, and even more preferably for approximately 2 h, to give the total or partial elimination of sulphate groups at position 2 of the glucosamine residues;

b) N-acylation of said totally or partially desulphated groups at position 2 of the glucosamine residues by treatment in alkaline aqueous solution (pH 8-9) with an acylating agent, such as acyl anhydrides, to give totally or partially acylated groups at position 2 of the glucosamine residues; then submitting the obtained compounds to steps c), d) or e) and f-g) below, or alternatively directly to step f) below;

c) basic treatment at a temperature ranging from ambient temperature to approximately 100° C., preferably from 50 to 70° C., and even more preferably at approximately 65° C., which leads to the elimination of a controlled percentage of sulphate groups in position 2 of the iduronic acid and to the formation of epoxide groups; and, if desired

d) opening of said epoxide ring at approximately pH 7, at a temperature ranging from approximately 50° C. to approximately 100° C., preferably at approximately 70° C., to yield residues of galacturonic acid; or, alternatively

e) opening of said epoxide ring at a temperature ranging from approximately 0° C. to 30° C., preferably at approximately 25° C., to yield residues of iduronic acid; and, if desired

f) oxidation of the diols with sodium periodate, to yield the opening of the glycoside ring and the formation of two aldehyde groups per modified residue; and, if desired;

g) reduction of said aldehyde groups to primary alcohol and, if desired, transformation of the D group to a group other than hydroxyl, as envisaged in the different meanings assigned in formula (I);

h) optional acid hydrolysis of compounds obtained in step g) to obtain oligosaccharides corresponding to the regular sequences, preferably by deamination with nitrous acid. This reaction, which is usually applied to obtain LMW heparin by cleaving the linkage between N-sulphate glucosamine residues and the next uronic acid, leads to a LMW compound having at the non reducing end a residue consisting of an uronic acid and at the reducing end a residue of anhydro mannose, this latter can be further modified to anhydromannitol by reduction with borohydride. The obtained LMW compounds contain at least one residue of glycol-split iduronic acid; or alternatively

i) submitting the products obtained in step g) to partial enzymatic hydrolysis with an enzyme selected from the group consisting of lyase, heparinase, heparitinase, or equivalent of to yield oligosaccharides, preferably tetra- or octa-saccharides, with the non-reducing terminal residue consisting of unsaturated iduronic acid, the reducing residue consisting of an N-sulphoglucosariine and containing at least one residue of open iduronic acid.

i) optionally the compound obtained in step c) or the product obtained in step d) is treated by partial enzme hydrolysis; and, if desired

j) subjection of the products obtained in one of steps b), c), and f) to partial 6-O-desulphation; or, alternatively,

k) subjection of the starting heparin partially or totally 6-desulphated to steps b), c) and f).

The 2-O-desulphated derivatives according to the present invention are obtained with the process above disclosed by omitting steps a) and b).

The process according to the present invention is also illustrated by the schemes below:

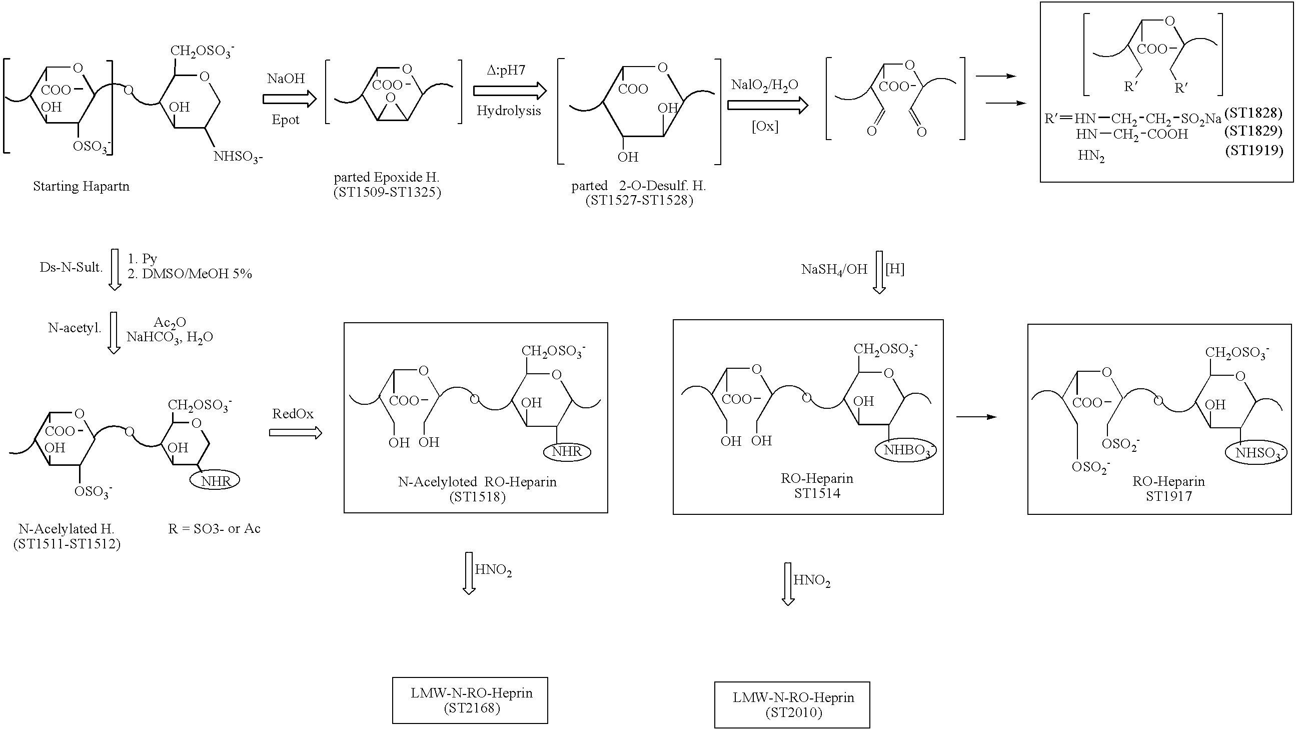

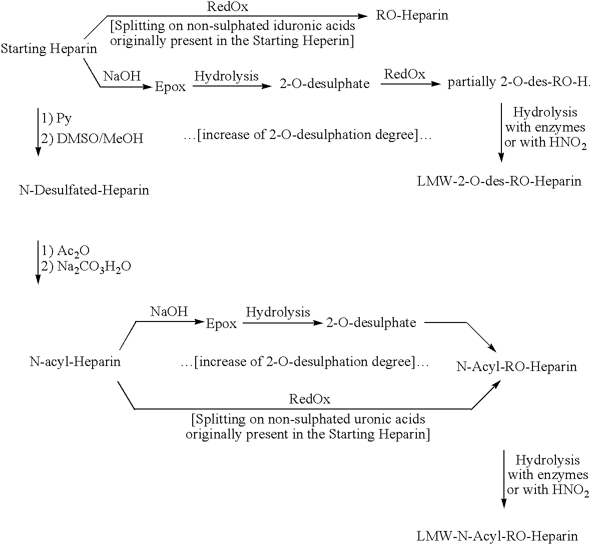

According to the invention described herein, the preferred compound are:

heparin partially 2-O-desulphated, obtainable by the process described above, where steps a) and b) are omitted, step c) is conducted for 45 min at 60° C., and step d) at 70° C. at pH 7, and having a molecular weight (MW) of 11200, a polydispersion index D of 1.3, a desulphation degree of 1.99 (expressed as the SO₃—:COO— molar ratio), percentage of modified uronic acid compared to total uronic acid of approximately 50% (hereinafter also called ST1514);

LMW heparin partially 2-O-desulphated, obtainable by the process described above, where steps a) and b) are omitted, step c) is conducted for 45 min at 60° C. and step d) at 70° C. at pH 7, followed by step f) g) and h) conducted by deamination and having a molecular weight (MW) of 3050, a polydispersion index D of 2.2, a desulphation degree of 1.99 (expressed as the SO₃—:COO— molar ratio), a percentage of modified uronic acid compared to total uronic acid of approximately 50% (hereinafter also called ST2010);

LMW heparin partially 2-O-desulphated, obtainable by the process described above, where steps a) and b) are omitted, step c) is conducted for 45 min at 60° C. and step d) at 70° C. at pH 7, followed by step f) g) and h) conducted by deamination and having a molecular weight Mn=5800, Mw=7520, a polydispersion index D of 1.294, a percentage of modified uronic acid compared to total uronic acid of approximately 50% (hereinafter also called ST2184);

heparin N-acetyl (50%), obtainable by the process described above, where step a) is conducted for 2 h at room temperature and step b) for 2 h at 4° C., steps c), d), e) are omitted, step f is conducted at 4° C. for one night, step g) for 3 h at room temperature and having a molecular weight (MW) of 11200, a polydispersion index D of 1.3, a desulphation degree of 1.6 (expressed as the SO₃—:COO— molar ratio), percentage of modified uronic acids compared to total uronic acids of approximately 30% (hereinafter also called ST1518).

LMW heparin N-acetyl (50%), obtainable by the process described above, where step a) is conducted for 2 h at room temperature and step b) for 2 h at 4° C., steps c), d), e) are omitted, step f is conducted at 4° C. for one night, step g) for 3 h at room temperature, step h) is conducted by nitrous acid deamination at 4° C. for 17 min, followed by reduction of aldehyde groups with borohydride at room temperature for 3 h, and having a molecular weight of Mw=4780, Mn=10000, a polydispersion index D of 2.092, a percentage of modified uronic acids compared to total uronic acids of approximately 30% (hereinafter also called ST2168).

heparin N-acetyl (27%), obtainable by the process described above, where step a) is conducted for 2 h at room temperature and step b) for 2 h at 4° C., steps c), d), e), f), g), h) are omitted, and having a molecular weight of Mn=10890, Mw=22370, a polydispersion index D of 2.054 (hereinafter also called ST2037).

heparin N-acetyl (39%), obtainable by the process described above, where step a) is conducted for 2 h at room temperature and step b) for 2 h at 4° C., steps c), d), e), f), g), h) are omitted, and having a molecular weight of Mn=10210, Mw=21270, a polydispersion index D of 2.083 (hereinafter also called ST2038).

heparin N-acetyl (64%), obtainable by the process described above, where step a) is conducted for 2 h at room temperature and step b) for 2 h at 4° C., steps c), d), e), f), g), h) are omitted, and having a molecular weight of Mn=11070, Mw=22000, a polydispersion index D of 1.987 (hereinafter also called ST2041).

heparin N-acetyl (27%), obtainable by the process described above, where step a) is conducted for 2 h at room temperature and step b) for 2 h at 4° C., steps c), d), e), f), g), h) are omitted, and having a percentage of modified uronic acids compared to total uronic acids of approximately 30% (hereinafter also called ST2185).

heparin N-acetyl (39%), obtainable by the process described above, where step a) is conducted for 2 h at room temperature and step b) for 2 h at 4° C., steps c), d), e), f), g), h) are omitted, and having a percentage of modified uronic acids compared to total uronic acids of approximately 30% (hereinafter also called ST2186).

heparin N-acetyl (64%), obtainable by the process described above, where step a) is conducted for 2 h at room temperature and step b) for 2 h at 4° C., steps c), d), e), f), g), h) are omitted, and having a percentage of modified uronic acids compared to total uronic acids of approximately 30% (hereinafter also called ST2187).

The preparation of compounds ST1514, ST1513, ST1516 and ST1515 are specifically disclosed in WO 01/55221.

The molecular weights are determined by HPLC-GPC (high performance liquid chromatography—gel permeation chromatography). The desulphation degree is determined by conductimetry and the percentage of modified uronic acids by ¹³C-NMR.

MW is the molecular weight, and D is the polydispersion index expressed as MW/Mn.

According to the invention described herein, the starting products are glycosaminoglycans of various origins, preferably naturally occurring heparins. It is also possible to use chemically modified heparins with a percentage content of N,6 disulphate ranging from 0 to 100%. Starting from products with a different 6-O-sulphated glucosamine content, it is possible to modulate the length of the regular sequences between one open iduronic acid and another. The glycosaminoglycans according to the invention that present opening of the glycoside ring are conventionally called RO derivatives by those skilled in the field, meaning by this that the glycoside ring has been opened by means of an oxidation action, followed by a reduction (Reduction-Oxidation—RO). This opening of the glycoside ring is also conventionally called “glycol split”, so-called because of the formation of the two primary hydroxy present on the open ring. The compounds referred to herein will also be called “RO” or “Glycol Split” derivatives.

In a further embodiment of the invention described herein, the aldehydes and primary hydroxy derived from the opening reaction described above (“glycol split”) also lend themselves to the subsequent functionalisation. Therefore, formula (I) compounds may also bear equal or different groups, as defined above for X and X′, on the primary hydroxy deriving from glycol split, for example, oligosaccharide or peptide groups, ranging from a single saccharide or amino acid to more than one unit of length, preferably 2 or 3 units.

Formula (I) compounds where X and X′ are —CH₂OH can also be used as vehicles for other types of drugs, by means of suitable binding with the heparin portion which is capable of providing a stable bond in normal conditions of manufacture and storage of a formulated drug, which, however, releases the transported drug in the body, preferably in the vicinity of the target organ. Examples of drugs that can be transported are steroidal and non-steroidal anti-inflammatory drugs, corticosteroids, and other drugs with an antimetastatic action, in which case there will be an advantageous enhancement of the antimetastatic effect as a result of the sum of the separate intrinsic activities of the compounds according to the invention and the antimetastatic agent bound thereto, with the related advantages of a greater target selectivity and lower systemic toxicity. Examples of these drugs are the metalloproteinase inhibitors. Other drugs which can be usefully transported are those that act at the endothelial level. Formula (I) compounds where X and X′ are other than hydroxy or aldehyde can also be used as vehicles for drugs, in which case the X and X′ groups will act as “spacers” between the transported molecule, that is to say the glycosaminoglycan of the present invention and the molecule acting as the vehicle, in those cases where this may be desirable for reasons of pharmacokinetics or pharmacodynamics.

In the case of compounds according to the invention deriving from heparin, these are prepared starting from heparin as such by means of N-desulphation followed by N-acylation using techniques known to the technical experts in the field. For example, the N-desulphation is conducted by solvolysis in DMSO:H₂O solution 95:5 v:v at room temperature for time ranging from 0.5 to 8 h followed by N-acylation in alkaline condition with, for example, acylanhydrides (i.e., acetyl, hexanoyl, succinyl, pivaloyl).

The following 2-O-desulphation is conducted in the presence of alkaline agents, such as sodium hydroxide, at temperatures ranging from ambient temperature to 100° C., preferably from 50 to 70° C., for example at 60° C., for a sufficiently long period to obtain the desired 2-O-desulphation. The 2-O-desulphation is controlled by acting on the process parameters, such as the concentrations of reactants, the temperature and the reaction times. One preferred example consists in maintaining constant concentrations of substrate (glycosaminoglycan) at 80 mg/ml and of NaOH at 1 M, a constant temperature of 60° C. and controlling the desulphation with a reaction time from 15 to 60 min. The expert in the field may vary the conditions, for example by raising the reaction temperature and shortening the reaction time, on the basis of normal trial and error in experimental practice and on the basis of his or her general knowledge of the subject.

The treatment with alkaline agents gives rise to an intermediate product characterised by the presence of an epoxide ring on the desulphated unit. In a thoroughly surprising manner, these intermediates have proved to be endowed with heparanase inhibiting properties similar to those of the formula (I) compounds. Therefore, a further object of the invention described herein is a derivative of partially 2-O-desulphated heparin, and therefore heparin with a reduced charge, particularly heparin not 2-O-desulphated more than 60%, characterised by an epoxide ring on the desulphation site. Said compounds characterised by an epoxide ring also belong to the whole scope covered by the present invention.

Subsequent to the formation of the epoxide ring, the latter is opened, again resorting to known techniques. The percentage of epoxide formed is calculated from the ratio between the areas of the ¹³C-NMR signals at approximately 55 ppm, characteristic of carbons 2 and 3 of the uronic acid ring containing the epoxide and the total number of anomeric signals (C1 of the glucosamine and uronic acid residues). If the opening is conducted hot, a galacturonic acid residue is obtained, whereas, if the opening of the epoxide ring is conducted cold, an iduronic acid residue is obtained. Preferred examples of compounds containing an epoxide ring are those obtainable by the process described above and having epoxidated uronic acid contents of 14% (hereinafter ST1509), 24% (hereinafter ST1525) and 30% (hereinafter ST1526), respectively.

The partially desulphated heparin is then subjected to “glycol-split” (RO for short), according to the process defined above and Smith degradation (SD for short).

Alternatively, formula (I) compounds can also be obtained without passing via the epoxide intermediate, that is to say by direct glycol split and subsequent Smith degradation.

The process described so far leads to formula (I) compounds in which the X and X′ groups are both —CH₂OH.

For X and X′ other than —CH₂OH, methods are available to the is expert in the field for transforming the hydroxyl group with other groups envisaged in the definitions given above (see for example Scheme on page 26, compounds ST1828, ST1829, ST1917 and ST1919). For example, the conjugation with amino acids or peptides can be done by treating the intermediate aldehyde derived from the glycol-split reaction with a reductive amination reaction (Hoffmann J. et al. Carbohydrate Research, 117, 328-331 (1983)), which can be conducted in aqueous solvent and is compatible with maintenance of the heparin structure.

If desired, and this constitutes a further object of the invention described herein, the formula (I) compounds can be further degraded with acid agents in suitable pH conditions, e.g. at pH 4, to yield a mixture of oligosaccharides that maintain the antiangiogenic properties.

In the same way, objects of the present invention are the compounds obtained by one of the steps g), h), i) and j) of the process described above.

Objects of the invention described herein are pharmaceutical compositions containing as their active ingredient at least one formula (I) compound, alone or in combination with one or more formula (I) compounds, or, said formula (I) compound or compounds in combination with the N-acyl-desulphated heparins described above, e.g. the epoxidated intermediates; the latter can also be used alone as active ingredients in the pharmaceutical compositions. The active ingredient according to the present invention will be in a mixture with suitable vehicles and/or excipients commonly used in pharmaceutical technology, such as, for instance, those described in “Remington's Pharmaceutical Sciences Handbook”, latest edition. The compositions according to the present invention will contain a therapeutically effective quantity of the active ingredient. The doses will be determined by the expert in the field, e.g. the clinician or primary care physician according to the type of disease to be treated and the patient's condition, or concomitantly with the administration of other active ingredients. By way of an example, doses ranging from 0.1 to 100 mg/kg may be indicated.

Examples of pharmaceutical compositions are those that can be administered orally or parenterally, intravenously, intramuscularly, subcutaneously, transdermally or in the form of nasal or oral sprays. Pharmaceutical compositions suitable for the purpose are tablets, hard or soft capsules, powders, solutions, suspensions, syrups, and solid forms for extemporary liquid preparations. Compositions for parenteral administration are, for example, all the intramuscular, intravenous and subcutaneous injectable forms as well as solutions, suspensions and emulsions. Liposome formulations should also be mentioned. The tablets also include forms for the controlled release of the active ingredient whether as oral administration forms, tablets coated with suitable layers, microencapsulated powders, complexes with cyclodextrins, depot forms, for example, subcutaneous forms, such as depot injections or implants.

The compounds according to the invention described herein possess anti-heparanase and antiangiogenic activity. This makes them suitable for the preparation of medicaments useful for the treatment of subjects, generally mammals, and particularly human subjects, suffering from altered angiogenesis or subjects who need a treatment inhibiting the heparanasic activity.

Examples of diseases treated with the medicament which is the object of the present invention are primary tumours, metastases, diabetic retinopathies, psoriasis, retrolenticular fibroplasia, restenosis after angioplasty, coronary by-pass, inflammation, arthritis, autoimmune diseases, allograft rejection, cardiovascular diseases, fibro-proliferative disease, diseases elicited by abnormal platelet aggregation, diseases elicited by smooth muscle proliferation, Goodpasture syndrome, acute glomerulonephritis, neonatal pulmonary hypertension, asthma, congestive heart failure, adult pulmonary hypertension, renal vascular hypertension, proliferative retinopathies, experimental autoimmune encephalomyelitis, multiple sclerosis, insulin dependent diabetes, inflammatory bowel disease, ulcerative colitis, Crohn's disease.

Advantageously, the compounds according to the present invention are substantially devoid of the side effects typical of heparin. In particular, the compounds according to the invention are substantially devoid of anticoagulant activity. By substantially devoid of such activity the expert in the field means no or only negligible activity from the point of view of clinical use.

The heparanase inhibiting activity was determined according to a method established by Vlodavsky's group (Bitan M. et al, 1995). The method is based on evaluation of the extent of fragmentation of the heparan sulphate chains of heparan sulphate proteoglycans (HSPG) caused by heparanase. Sulphate labelled extracellular matrix (ECM) is most commonly used as a source of HSPG. Sulphate labelled ECM is incubated with recombinant heparanase at pH 6.2 in the absence and in the presence of increasing concentrations of the test compound. To evaluate the occurrence of proteoglycan degradation, the incubation medium is collected and applied for gel filtration on Sepharose 6B columns (0.9×30 cm). Fractions (0,2 ml) are eluted with PBS at a flow rate of 5 ml/h and counted for radioactivity. The excluded volume (Vₒ) is marked by blue dextran and the total included volume (Vₜ) by phenol red. Degradation fragments of HS side chains are eluted from Sepharose 6B at 0.5<Kₐᵥ<0.8 (peak II). Under the reported experimental conditions, good heparanase inhibitors inhibit fragmentation of HS at concentrations of 10 μg/ml or less.

The results are shown in Table 1, below.

TABLE 1
Heparanase inhibition at dose ranging from 25 μg/ml to 5
μg/ml

| | | Inhibition | | |
|---|---|---|---|---|
| | Dose | 25 μg/ml | 10 μg/ml | 5 μg/ml |
| | Heparin | 100% | n.d. | >100% |
| ST1516 | Heparin 40% RO | 100% | n.d. | >85% |
| ST1514 | Heparin ˜50% RO | 100% | 100% | >85% |
| ST1515 | Heparin 27.5% RO | 100% | 100% | 100% |
| ST1518 | 50% NAc heparin | 100% | 100% | >85% |
| | 30% RO | | | |

Worthy to be noted, ST1518 has a high inhibition activity even at the concentration of 1 μg/ml.

The compounds according to the present invention, and in particular new one, were tested for their activity with respect to FGF's growth factors, with the same experimental model as described in WO 01/55221 and showed an activity comparable with the ones disclosed in the cited reference.

The following examples further illustrate the invention.

### EXAMPLE 1

ST1518

An excess of pyridine was added to an aqueous solution of 1 g of heparin, previously eluted from a column of Amberlite IR 120. The solution was evaporated under reduced pressure; the resulting pyridine salt of the heparin was dissolved in 50 ml of a mixture of DMSO/H₂O 95:5 and stirred at 20° C. for 2 hours, in order to obtain a desulphation degree of about 50%.

Then, the solution was diluted with an equal volume of a saturated solution of NaHCO₃. The solution was dialysed against distilled water in membranes (cut-off 1000-2000D). The final product was isolated by evaporation under reduced pressure.

N-acetylated heparin was prepared by N-acetylation of 50% N-desulphated heparin. 1 g of heparin was dissolved in 10 ml of distilled water; the solution was cooled to 4° C. and saturated with sodium hydrogen carbonate; 625 μl of acetic anhydride were added to this solution and the mixture was stirred for 2 hours at 4° C. During the reaction, pH was controlled and maintained at about 8 by adding sodium hydrogen carbonate. Then, the solution obtained was dialysed against distilled water in membranes (cut-off 2000-1000 D).

1 g of heparin 50% N-acetylated heparin is dissolved in 25 ml of distilled water and cooled to 4° C. after the addition of 25 ml of a solution of NaIO₄ 0.2 M, the solution is left to stir in the dark for 20 hours, and the reaction is stopped by adding ethylene glycol and the salts are eliminated by tangential ultrafiltration. 400 mg of NaBH₄, subdivided in several portions, are added to the desalted solution. The solution is left to stir for 3 hours at ambient temperature, then neutralized with diluted HCl and desalted by tangential ultrafiltration.

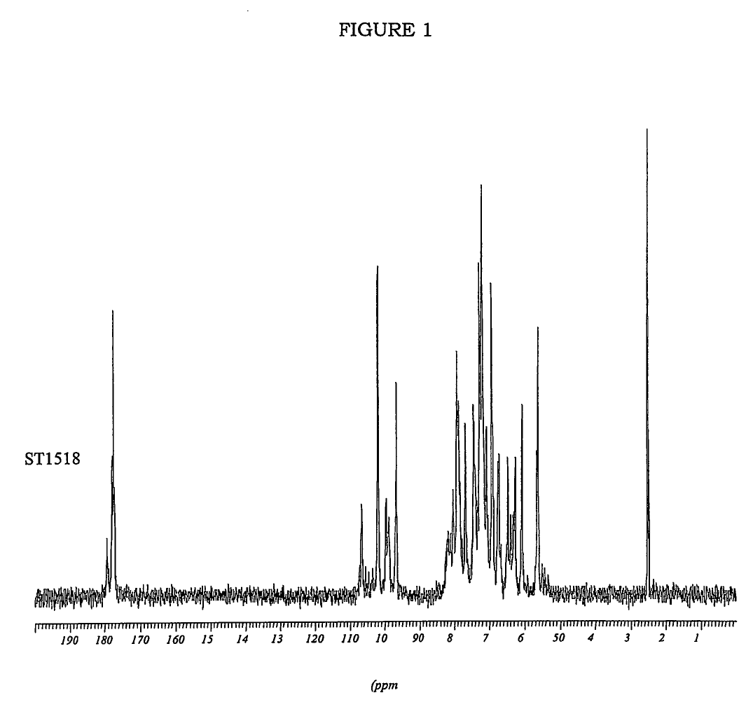

The ¹³C NMR spectrum of the compound is shown in FIG. 1.

### EXAMPLE 2

ST2010 and ST2184

5 g of heparin is dissolved in 63 ml of a solution NaOH 1N. The solution is left to stir for 45 min at 60° C., cooled and neutralized with diluted HCl. Then, the solution was stirred for 48 h at 70° C., cooled and dialysed against water in membranes (cut-off 2000-1000 D).

2 g of 2-O-desulphated heparin is dissolved in 50 ml of distilled water and cooled to 4° C. after the addition of 50 ml of a solution of NaIO₄ 0.2 M, the solution is left to stir in the dark for 20 hours, and the reaction is stopped by adding ethylene glycol and the salts are eliminated by tangential ultrafiltration. 800 mg of NaBH₄, subdivided in several portions, are added to the desalted solution. The solution is left to stir for 3 hours at ambient temperature, then neutralized with diluted HCl and desalted by tangential ultrafiltration.

400 mg of oxidated-reducted heparin are dissolved in 25 ml of distilled water. After the addition of 7 mg NaNO₂, the pH is adjusted to 2 with diluted HCl, and the solution is left to stir for 17 min at 4° C. The reaction is stopped by neutralization. 60 mg of NaBH₄, subdivided in several portions, are added to the desalted solution. The solution is left to stir for 3 hours at ambient temperature, then neutralized with diluted HCl and fractionated by gel filtration. Two fractions with different molecular weights were isolated: ST2010 having a Mw=3050 and ST2184 having a Mn=5800, Mw=7520.

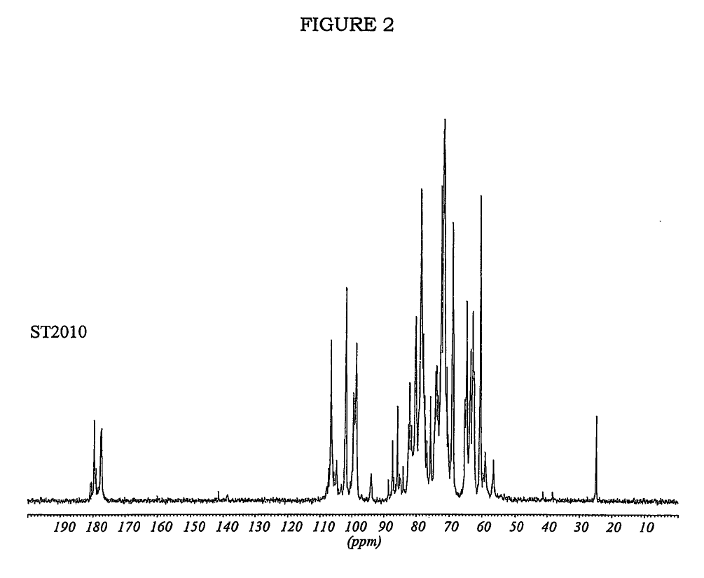

The ¹³C NMR spectrum of the compound ST2010 is shown in FIG. 2.

### EXAMPLE 3

ST2041

An excess of pyridine was added to an aqueous solution of 2 g of heparin, previously eluted from a column of Amberlite IR 120. The solution was evaporated under reduced pressure; the resulting pyridine salt of the heparin was dissolved in 100 ml of a mixture of DMSO/H₂O 95:5 and stirred at 20° C. for 4 hours, in order to obtain a desulphation degree of about 64%.

Then, the solution was diluted with an equal volume of a saturated solution of NaHCO₃. The solution was dialysed against distilled water in membranes (cut-off 1000-2000D). The final product was isolated by evaporation under reduced pressure.

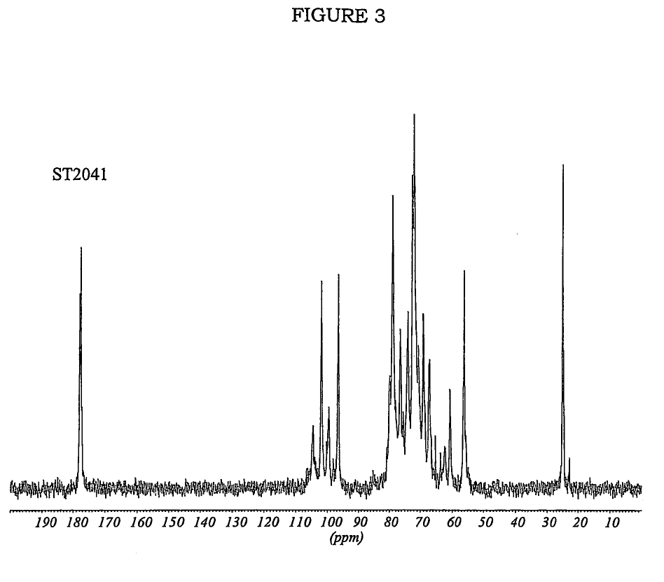

The ¹³C NMR spectrum of the compound ST2041 is shown in FIG. 3.

1. Use of a glycosaminoglycan derivative, particularly a desulphated heparin, with a desulphation degree not greater than 60% of the total uronic units for the preparation of a medicament having heparanase inhibiting activity and/or FGF growth factor inhibiting activity.

2. Use according to claim 1, wherein said derivative is a heparin-like glycosaminoglycan.

3. Use according to claim 1, wherein said derivative is a modified heparin, containing glycosamine residues with different degrees of N-desulphation and optional subsequent total or partial N-acylation.

4. Use according to claim 1, wherein said derivative has the following formula (I): 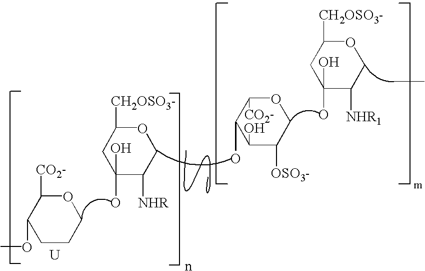
where the U ring can have the following meanings: 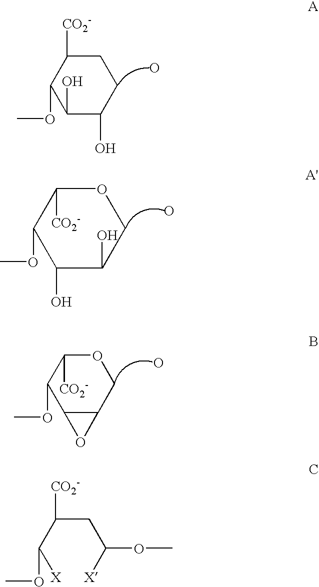
X and X′, which can be the same or different, are an aldehyde group or the —CH₂—D group, where D is hydroxy or an amino acid, a peptide or a residue of a carbohydrate or oligosaccharide;
R and R₁, which can be the same or different, are an SO₃, a C₁-C₈ acyl residue, optionally bearing at least a further carboxy group;
n and m, which can be the same or different, may vary from 1 to 40; the sum of n+m ranges from 6 to 40; the m:n ratio ranges from 10:2 to 1:1, the symbol [symbol] indicates that the units marked m and n are statistically distributed along the polysaccharide chain and are not necessarily in sequence.

5. Use of a derivative according to claim 4, wherein R or R₁ are N-acyl groups and range from 40 to 60% of the sum R+R₁.

6. Use according to claim 4, wherein R and R₁, which can be the same or different, are acetyl.

7. Use of a derivative according to claim 4, wherein m is greater than or equal to n.

8. Use of a derivative according to claim 5, wherein n ranges from 40 to 60% of the sum m+n.

9. Use according to claim 4, wherein said derivative is selected from the group consisting of:
partially 2-O-desulphated heparin with a molecular weight (MW) of 11200, a polydispersion index D of 1.3, a desulphation degree of 1.99 (expressed as SO₃—:COO— molar ratio), percentage of modified uronic acids compared to total uronic acids approximately 50%, m:n=1:1 and the units marked m and n are distributed along the polysaccharide chain in a regular, alternating manner;
LMW heparin partially 2-O-desulphated with a molecular weight (MW) of 3050, a polydispersion index of 2.2, a desulphation degree of 1.99 (expressed as the SO₃—:COO— molar ratio)), a percentage of modified uronic acids compared to total uronic acids of approximately 50%, m:n=1:1 and the units marked m and n are distributed along the polysaccharide chain in a regular, alternating manner;
LMW heparin partially 2-O-desulphated with a molecular weight of Mn=5800, Mw=7520, a polydispersion index of 1.294, a percentage of modified uronic acids compared to total uronic acids of approximately 50%, m:n=1:1 and the units marked m and n are distributed along the polysaccharide chain in a regular, alternating manner;
partially 2-O-desulphated heparin with a molecular weight (MW) of 12900 D, a polydispersion index D of 1.5, a desulphation degree of 1.9 (expressed as SO₃—:COO— molar ratio), percentage of modified uronic acids compared to total uronic acids: 5% epoxide groups, 29% oxidated and reduced uronic residues m:n=1:1 and the units marked m and n are distributed along the polysaccharide chain in a regular, alternating manner;
partially 2-O-desulphated heparin with a molecular weight (MW) of 9200 D, a polydispersion index D of 1.5, percentage of modified uronic acids compared to total uronic acids: 11% epoxide groups, 27.5% oxidated and reduced uronic residues, m:n=1:1 and the units marked m and n are distributed along the polysaccharide chain in a regular, alternating manner;
2-O-desulphated heparin with a molecular weight (MW) of 11000 D, a polydispersion index D of 1.5, a desulphation degree of 1.93 (expressed as SO₃—:COO— molar ratio), a percentage of modified uronic acids compared to total uronic acids: 5% epoxide groups, 29% oxidated and reduced uronic residues.

10. Use according to claim 4, wherein said derivative is selected from the group consisting of:
partially N-desulphated and N-reacetylated heparin with a molecular weight (MW) of 11250 a polydispersion index of 1.66, a desulphation degree of 1.7 (expressed as the SO₃—:COO— molar ratio)), a percentage of modified uronic acids compared to total uronic acids of approximately 30%, the 50% of the sum of R and R₁ is N-acetyl;
partially N-desulphated and N-reacetylated LMW heparin with a molecular weight of Mn=4780, Mw=10000, a polydispersion index D of 2.092, a percentage of modified uronic acids compared to total uronic acids of approximately 30%, the 50% of the sum of R and R₁ is N-acetyl;
partially N-desulphated and N-reacetylated heparin with a molecular weight of Mn=10890, Mw=22370, a polydispersion index of 2.054, the 27% of the sum of R and R₁ is N-acetyl;
partially N-desulphated and N-reacetylated heparin with a molecular weight of Mn=10210, Mw=21270, a polydispersion index of 2.083, the 39% of the sum of R and R₁ is N-acetyl;
partially N-desulphated and N-reacetylated heparin with a molecular weight of Mn=11070, Mw=22000, a polydispersion index of 1.987, the 64% of the sum of R and R₁ is N-acetyl;
partially N-desulphated and N-reacetylated heparin, a percentage of modified uronic acids compared to total uronic acids of approximately 30%, the 27% of the sum of R and R₁ is N-acetyl;
partially N-desulphated and N-reacetylated heparin, a percentage of modified uronic acids compared to total uronic acids of approximately 30%, the 39% of the sum of R and R₁ is N-acetyl;
partially N-desulphated and N-reacetylated heparin, a percentage of modified uronic acids compared to total uronic acids of approximately 30%, the 64% of the sum of R and R₁ is N-acetyl.

11. Use according to claim 1, wherein said medicament has antiangiogenic activity.

12. Use according to claim 1, wherein said medicament is useful for the treatment of inflammations.

13. Use according to claim 1, wherein said medicament is useful for the treatment of autoimmune diseases.

14. Use according to claim 1, wherein the disease to be treated is selected from the group consisting of primary tumours, metastases, diabetic retinopathies, psoriasis, retrolenticular fibroplasia, restenosis after angioplasty, coronary by-pass, inflammation, arthritis, autoimmune diseases, allograft rejection, cardiovascular diseases, fibro-proliferative disease, diseases elicited by abnormal platelet aggregation, diseases elicited by smooth muscle proliferation, Goodpasture syndrome, acute glomerulonephritis, neonatal pulmonary hypertension, asthma, congestive heart failure, adult pulmonary hypertension, renal vascular hypertension, proliferative retinopathies, multiple sclerosis, experimental autoimmune encephalomyelitis, insulin dependent diabetes, inflammatory bowel disease, ulcerative colitis, Crohn's disease.

15. A compound of formula (I) 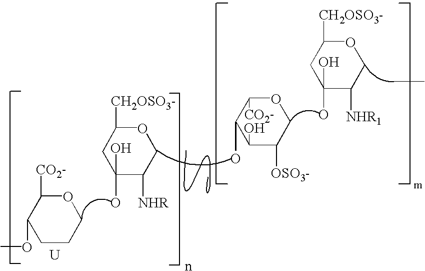
where the U ring can have the following meanings: 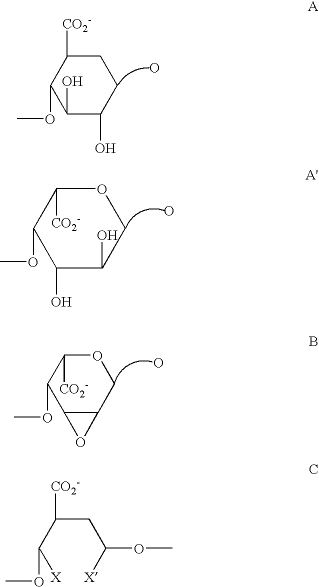
X and X′, which can be the same or different, are an aldehyde group of the —CH₂—D group, where D is hydroxy or an amino acid, a peptide or a residue of a carbohydrate or oligosaccharide;
R and R₁, which can be the same or different, are an SO₃, a C₁ or C₃-C₈ acyl residue;
n and m, which can be the same or different, may vary from 1 to 40; the sum of n+m ranges from 6 to 40; the m:n ratio ranges from 10:2 to 1:1, the symbol [symbol] indicates that the units marked m and n are statistically distributed along the polysaccharide chain and are not necessarily in sequence.

16. Compound according to claim 15, selected from the group consisting of:
partially N-desulphated and N-reacetylated heparin with a molecular weight (MW) of 11250, a polydispersion index of 1.66, a desulphation degree of 1.7 (expressed as the SO₃—:COO— molar ratio), a percentage of modified uronic acids compared to total uronic acids of approximately 30% and the 50% of the sum of R and R₁ is N-acetyl;
LMW partially N-desulphated and N-reacetylated heparin with a molecular weight of Mn=4780, Mw=10000, a polydispersion index of 2.092, a percentage of modified uronic acids compared to total uronic acids of approximately 30%, the 50% of the sum of R and R₁ is N-acetyl;
partially N-desulphated and N-reacetylated heparin with a molecular weight of Mn=10890, Mw=22370, a polydispersion index of 2.054, the 27% of the sum of R and R₁ is N-acetyl;
partially N-desulphated and N-reacetylated heparin with a molecular weight of Mn=10210, Mw=21270, a polydispersion index of 2.083, the 39% of the sum of R and R₁ is N-acetyl;
partially N-desulphated and N-reacetylated heparin with a molecular weight of Mn=11070, Mw=22000, a polydispersion index of 1.987, the 64% of the sum of R and R₁ is N-acetyl;
partially N-desulphated and N-reacetylated heparin, a percentage of modified uronic acids compared to total uronic acids of approximately 30%, the 27% of the sum of R and R₁ is N-acetyl.

17. Process for the preparation of the compound of claim 15, comprising the following steps:
a) N-desulphation by solvolytic hydrolysis of sulphamino residues in DMSO:H₂O 95:5 v:v at ambient temperature for a time ranging from 0.5 to 8 h, to give the total or partial elimination of sulphate groups at position 2 of the glucosamine residues;
b) N-acylation of said totally or partially desulphated groups at position 2 of the glucosamine residues by treatment in alkaline aqueous solution (pH 8-9) with an acylating agent, to give totally or partially acylated groups at position 2 of the glucosamine residues; then submitting the obtained compounds to steps c), d) or e) and f-g) below, or alternatively directly to step f) below;
c) basic treatment at a temperature ranging from ambient temperature to approximately 100° C., which leads to the elimination of a controlled percentage of sulphate groups in position 2 of the iduronic acid and to the formation of epoxide groups; and, if desired
d) opening of said epoxide ring at approximately pH 7, at a temperature ranging from approximately 50° C. to approximately 100° C., to yield residues of galacturonic acid; or, alternatively
e) opening of said epoxide ring at a temperature ranging from approximately 0° C. to 30° C., to yield residues of iduronic acid; and, if desired
f) oxidation of the diols with sodium periodate, to yield the opening of the glycoside ring and the formation of two aldehyde groups per modified residue;
g) reduction of said aldehyde groups to primary alcohol and, if desired, transformation of the D group to a group other than hydroxyl, as envisaged in the different meanings assigned in formula (I);
h) optional acid hydrolysis of compounds obtained in step g) to obtain oligosaccharides corresponding to the regular sequences; or alternatively
i) submitting the products obtained in step g) to partial enzymatic hydrolysis with an enzyme selected from the group consisting of lyase, heparinase, heparitinase, or equivalent of to yield oligosaccharides, preferably tetra- or octa-saccharides, with the non-reducing terminal residue consisting of unsaturated iduronic acid, the reducing residue consisting of an N-sulphoglucosamine and containing at least one residue of open iduronic acid;
j) optionally the compound obtained in step c) or the product obtained in step d) is treated by partial enzyme hydrolysis; and, if desired
k) subjection of the products obtained in one of steps b), c), and f) to partial 6-O-desulphation; or, alternatively,
l) subjection of the starting heparin partially or totally 6-desulphated to steps b), c) and f).

18. Compounds obtainable by the process of claim 17.

19. Compounds obtainable by the process of claim 17, selected from the group consisting of:
heparin N-acetyl (50%), where step a) is conducted for 2 h at room temperature and step b) for 2 h at 4° C., steps c), d), e) are omitted, step f) is conducted at 4° C. for one night, step g) for 3 h at room temperature and having a molecular weight (MW) of 11200, a polydispersion index D of 1.3, a desulphation degree of 1.6 (expressed as the SO₃—:COO— molar ratio), percentage of modified uronic acids compared to total uronic acids of approximately 50%;
LMW heparin N-acetyl (50%), where step a) is conducted for 2 h at room temperature and step b) for 2 h at 4° C., steps c), d), e) are omitted, step f) is conducted at 4° C. for one night, step g) for 3 h at room temperature, step h) is conducted by nitrous acid deamination at 4° C. for 17 min, followed by reduction of aldehyde groups with borohydride at room temperature for 3 h, and having a molecular weight of Mw=4780, Mn=10000, a polydispersion index D of 2.092, a percentage of modified uronic acids compared to total uronic acids of approximately 30%;
heparin N-acetyl (27%), where step a) is conducted for 2 h at room temperature and step b) for 2 h at 4° C., steps c), d), e), f), g), h) are omitted, and having a molecular weight of Mn=10890, Mw=22370, a polydispersion index D of 2.054;
heparin N-acetyl (39%), where step a) is conducted for 2 h at room temperature and step b) for 2 h at 4° C., steps c), d), e), f), g), h) are omitted, and having a molecular weight of Mn=10210, Mw=21270, a polydispersion index D of 2.083;
heparin N-acetyl (64%), where step a) is conducted for 2 h at room temperature and step b) for 2 h at 4° C., steps c), d), e), f), g), h) are omitted, and having a molecular weight of Mn=11070, Mw=22000, a polydispersion index D of 1.987;
heparin N-acetyl (27%), where step a) is conducted for 2 h at room temperature and step b) for 2 h at 4° C., steps c), d), e), f), g), h) are omitted, and having a percentage of modified uronic acids compared to total uronic acids of approximately 30%;
heparin N-acetyl (39%), where step a) is conducted for 2 h at room temperature and step b) for 2 h at 4° C., steps c), d), e), f), g), h) are omitted, and having a percentage of modified uronic acids compared to total uronic acids of approximately 30%;
heparin N-acetyl (64%), obtainable by the process described above, where step a) is conducted for 2 h at room temperature and step b) for 2 h at 4° C., steps c), d), e), f), g), h) are omitted, and having a percentage of modified uronic acids compared to total uronic acids of approximately 30%.

20. Pharmaceutical compositions containing at least one compound of claim 15 as an active ingredient in admixture with pharmaceutically acceptable vehicles and excipients.

21. Use of compounds of claim 15 as medicaments.

22. Use of the compounds of claim 15 as vehicles for drugs.

23. Use according to claim 22, wherein said drug is selected from the group consisting of: steroidal and non-steroidal anti-inflammatory drugs, corticosteroids, drugs with antimetastatic action, drugs that act at the endothelial level.

24. Use according to claim 23, wherein said antimetastatic drug is a metalloproteinase inhibitor.
